(19) United States
(12) Patent Application Publication
(10) Pub. No.: US 2010/0331749 A1
(43) Pub. Date: Dec. 30, 2010

(54) ORTHOTIC HEEL SUPPORT DEVICE

(76) Inventor: Michael J. Powaser, Lodi, CA (US)

Correspondence Address: BULLIVANT HOUSER BAILEY PC, 1415 L STREET, SUITE 1000, SACRAMENTO, CA, 95814, US

(21) Appl. No.: 12/827,913

(22) Filed: Jun. 30, 2010

Related U.S. Application Data
(60) Provisional application No. 61/221,945, filed on Jun. 30, 2009.

(51) Int. Cl. A61F 5/00

(52) U.S. Cl. 602/23

(57) ABSTRACT

This invention provides an orthotic sock comprising a tubular main body having two ends, one of which may be sized to snugly fit over the wearer's ankle while the second end may be sized to snugly fit over at least a portion of the wearer's foot, an orthotic heel lift and an internal pocket, located within the main body, that substantially encloses a heel lift whereby the lift is held in place against the wearer's heel by the sock.

### REFERENCE TO RELATED APPLICATION

This application claims the benefit of U.S. Provisional Patent Application No. 61/221,945, filed Jun. 30, 2009, the complete disclosure of which is incorporated herein, in its entirety.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent files and records, but otherwise reserves all other copyright or related rights.

### BACKGROUND OF THE INVENTION

1. Field of the Invention

This invention relates to means for holding an orthotic heel support device in place in a situation where the wearer wishes to use sports footwear or no footwear. Described is a compression sock that holds an orthotic heel support device in place.

2. Description of Related Art

Millions of people suffer from orthopedic problems associated with the foot and spine. Various types of orthopedic support devices, such as heel lifts, are well known in the art. Orthopedic support devices, such as heel lifts, for those with one leg shorter than the other, are normally secured to the insoles of specially fashioned conventional shoes to maintain the correctly aligned position of the foot and spine. Generally, for those who need to use different types of shoes or no shoes, such as athletes, there are limited means of receiving the benefits of an orthopedic support device.

Various attempts have been made to allow the use of orthopedic support devices with or without shoes through the use of a harness or bandage that wraps around the foot. U.S. Pat. No. 6,558,339, issued to Graham on May 6, 2003 for “Foot Alleviator”, discloses a removable semi-elastic brace for alleviating heel and arch pain. The Graham device consists of a bandage configured to wrap around the foot with small pads affixed to the interior of the bandage. When wrapped around the foot, the bandage is held in place by Velcro fasteners. Although the Graham device succeeds in providing a harness that does not limit the wearer to a single pair of specially fashioned shoes, this device has several limitations. Hygienic issues may arise because the device is configured to allow direct contact between the pads and the wearer's foot. Moreover, it is not clear how the pads are secured to the bandage and, depending on how this is achieved, there may be the potential for slippage during use, depriving the wearer of the benefits of such pads. The device employs Velcro fasteners which, while effective when new, quickly wear out under the rigors of daily use in a sports setting and the bandage disclosed by Graham has many such fastening points where failure could occur. Finally, the pads disclosed in the Graham device are said to be intended to relieve stress on portions of the foot. An orthotic heel support device is directed toward a different problem, that of correcting foot positioning so as to avoid broader posture problems.

U.S. Pat. No. 6,059,744, issued to Hardt on May 9, 2009 for “Ankle Support and Heel Cushioning Device”, discloses an ankle strap device that provides ankle support and heel cushioning, which can be worn with or without a shoe. This device comprises a heel cup that is sewn or otherwise permanently affixed to a strap that encircles the wearer's ankle. Although this device avoids the potential for the failure of fasteners and likely avoids the problem of slippage, the Hardt device allows direct contact between the heel cup and the wearer's foot which could cause hygienic issues as the wearer's foot sweats. In addition, this device requires the preparation of an individualized heel cup that is permanently affixed to the securing strap. This device is, therefore, inherently more expensive to manufacture than one that can be easily modified for any patient's needs. As with the Graham device, the Hardt device is used for cushioning the heel rather than for orthotic support.

Sock and stocking devices, used to treat injury or circulatory ailments, are also well known to the art. Compression socks, also known as compression hoses or compression stockings, provide therapy by applying pressure from the ankle up the leg, for treatment of circulatory and venous disorders, small cosmetic varicosities to venous ulcers, and the like. This pressure compresses surface veins, keeping them small and forcing blood to deeper veins in the legs. It also reduces edema and swelling by accelerating the velocity of blood flow to the heart and ends pooling of blood, thus assuring healthier feet and legs. Compression socks can also provide support and stability to relieve shin splint pain.

U.S. Pat. No. 7,056,299, granted to Brown, et al. on Jun. 6, 2006 for “Device for Heel Shock Absorption, Swelling, and Pain Treatment”, discloses an orthotic device for absorption of heel shock, treating edema and swelling and for lessening pain. The device disclosed by Brown consists of a tubular sleeve enclosing the ankle and a portion of the foot with an opening at the heel and a pocket located under the heel formed by sewing a piece of fabric to the outside of the sleeve. A wedge shaped pad is placed into the pocket to assist in the absorption of heel shock. Although the Brown device alleviates the problematic direct contact between the foot and the pad and the potential for slippage of the pad, both seen in Graham device, the Brown device is still not an optimal solution. Because the pocket into which the pad is placed is on the outside of the sleeve, rather than on the inside, the device has an open flap that may bunch or snag while in contact with a shoe or allow for the dislodging of the pad. Likewise, the presence of an open heel and pocket on the exterior, provides edges that may bunch or become snagged, leading to wear on the device. These openings also render the device unsuitable for use without shoes. Finally, like the Graham and Hardt devices, the device disclosed by Brown is not provided as an orthotic corrective measure, but rather, as padding for the treatment of foot pain and swelling.

As demonstrated by the devices disclosed in these publications, despite a long felt need, none of the prior approaches have provided a simple, durable, hygienic and cost-effective invention for combining the benefits of a compression sock with a means of securely holding in place an orthopedic support device in a manner that allows for the use of various sports-type footwear or no footwear by the wearer.

### SUMMARY OF THE INVENTION

This invention provides an orthotic sock comprising a tubular main body having two ends, an orthotic heel lift and an internal pocket, located within the main body, which substantially encloses a heel lift. One of the ends of the sock may be sized to snugly fit over the wearer's ankle while the second end may be sized to snugly fit over at least a portion of the wearer's foot. The heel lift, which may be adjustable to accommodate the differing needs of patients, may be removably secured within the pocket and the pocket may be located so as to hold the orthotic heel lift against the wearer's heel. The heel lift may be composed of foam rubber and may be textured on its surface to prevent slippage. Either or both of the pocket and the tubular main body of the sock could be composed of an elastic material.

In one preferred embodiment, the tubular body has openings at both the end that is positioned over a portion of the foot and the end that is positioned over the ankle area. In alternative embodiments, the portion of the tubular body that is positioned opposite the foot, extends to about 1 inch along the ankle region, in another embodiment, the same end extends about 2 inches up the ankle, in another, it extends 3 inches and, in another, it extends over the entire ankle. In yet another embodiment the end opposite the foot extends over both the ankle and at least a portion of the wearer's calf. In various other embodiments, the main body of the sock is dimensioned to provide compression over the wearer's calf, foot and/or ankle The main body of the sock may also be constructed so as to provide support to the arch of the wearer's foot.

### BRIEF DESCRIPTION OF THE DRAWINGS

A more complete understanding of the present invention and the attendant features and advantages thereof may be had by reference to the following detailed description when considered in conjunction with the accompanying drawings wherein:

FIG. 1 shows a perspective view of the orthotic heel support device, with a pocket for inserting a heel lift device and a lift device in the pocket.

FIG. 2 is a side view of the orthotic heel support device, with a heel lift device included in the pocket.

FIG. 3 is a top view of the orthotic heel support device, with a heel lift device included in the pocket.

FIG. 4 is a front view of the orthotic heel support device, with a heel lift device included in the pocket.

### DETAILED DESCRIPTION OF THE INVENTION

The present invention provides a simple, durable, hygienic and cost-effective device that combines the benefits of a compression sock with a means of securely holding in place an orthopedic support device in a manner that allows for the use of various sports-type footwear or no footwear by the wearer. The device consists of a compression sock having an internal pocket at the heel into which is inserted a heel lift.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention.

In this description, the term “orthotic heel lift” means a device of any shape, size or composition that is intended to be placed under at least a portion of the heel region of the foot with the therapeutic objective of correctly aligning the foot.

By “adjustable,” when used in reference to the orthotic heel lift, is meant that the orthotic heel lift may be easily removed and replaced with a differently configured orthotic heel lift.

By “compression” is meant the relatively even application of pressure over a surface.

The term “elastic material” is intended to be broadly construed and includes any material that may be stretched and thereafter substantially resume the shape it had prior to stretching.

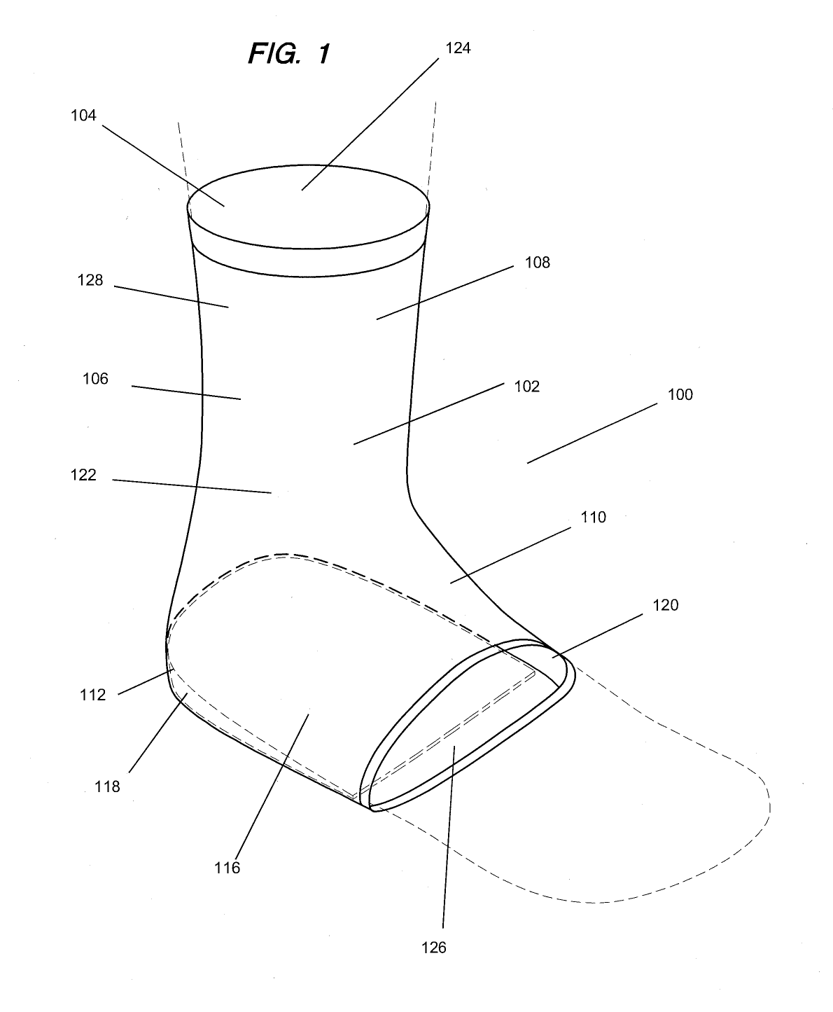

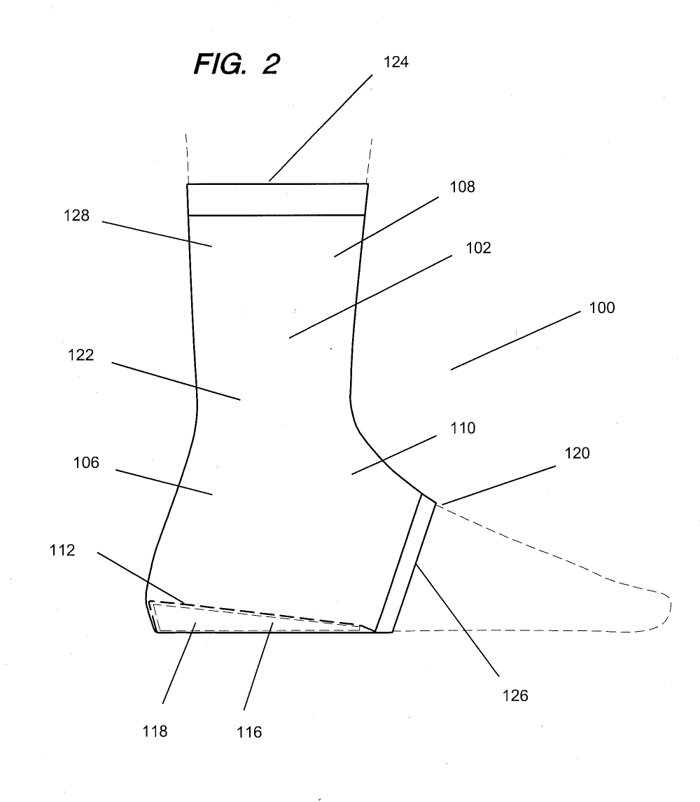

FIGS. 1 through 4 show one embodiment of the invention. As shown in FIGS. 1 and 2, in this embodiment, the orthotic sock 100 consists of a tubular main body 102 forming interior 104 and exterior surfaces 106, and having a first end 108 and a second end 110. The first end 108 extends over the wearer's entire ankle 122 and terminates at about the lower portion of the wearer's calf 128 where there is an opening 124. In alternative embodiments, the first end 108 may not extend as far or may extend further. The second end 110 extends over the middle portion of the wearer's foot 120 where there is an opening 126. In alternative embodiments, the second end 110 may extend to cover more of the foot or may cover the entire foot without an opening

As shown in FIGS. 1 through 4, both ends 108, 110 are sized to fit snugly over the portions of the foot and leg they cover and the tubular main body 102 may be composed of an elastic material to promote such a fit. At the heel of the tubular main body 102 is an flap of material 112 that forms and internal pocket 116 with the interior 104 of the sock 100. Various suitable means can be used in forming the pocket 116, for instance the pocket 116 could also be formed by sewing two separate pieces of fabric together on and, in turn, sewing the resulting structure to the interior 104 of the sock 100.

The internal pocket 116 is sized to accommodate a wedge shaped orthotic heel lift 118 that, in this embodiment, is primarily composed of a dense and durable foam rubber material. The orthotic heel lift 118 is also textured on one surface so as to retard slippage. The pocket is also sized and positioned so that the lift cannot slide about during use and is maintained in the correct position under the heel so as to continuously provide the intended orthopedic support.

As will be apparent from the drawings, the invention's configuration provides several benefits. The snugly fitting tubular main body 102, having the pocket 116 holding the orthotic heel lift 118 fitted internally, is advantageous because it serves to hold the orthotic heel lift 118 snugly in place against the wearer's heal. The continuous tubular main body 102 is continuous and smooth on the exterior around the heel, without having openings or flaps, ensuring that such openings or flaps do not bunch upon interaction with footwear or snag or catch other things should the wearer choose not to use shoes. At least one layer of fabric is interposed between the orthotic heel lift 118 and the wearer's heel, allowing for air circulation. Moreover, the orthotic heel lift 118 is removable. This configuration allows the wearer to separately wash the tubular main body 102 and the lift 118, insuring good hygiene. The removability of the lift 118 also allows for the easy exchange of different orthotic heel lifts, allowing the device to be quickly modified for use by any patient without significant expense or bother.

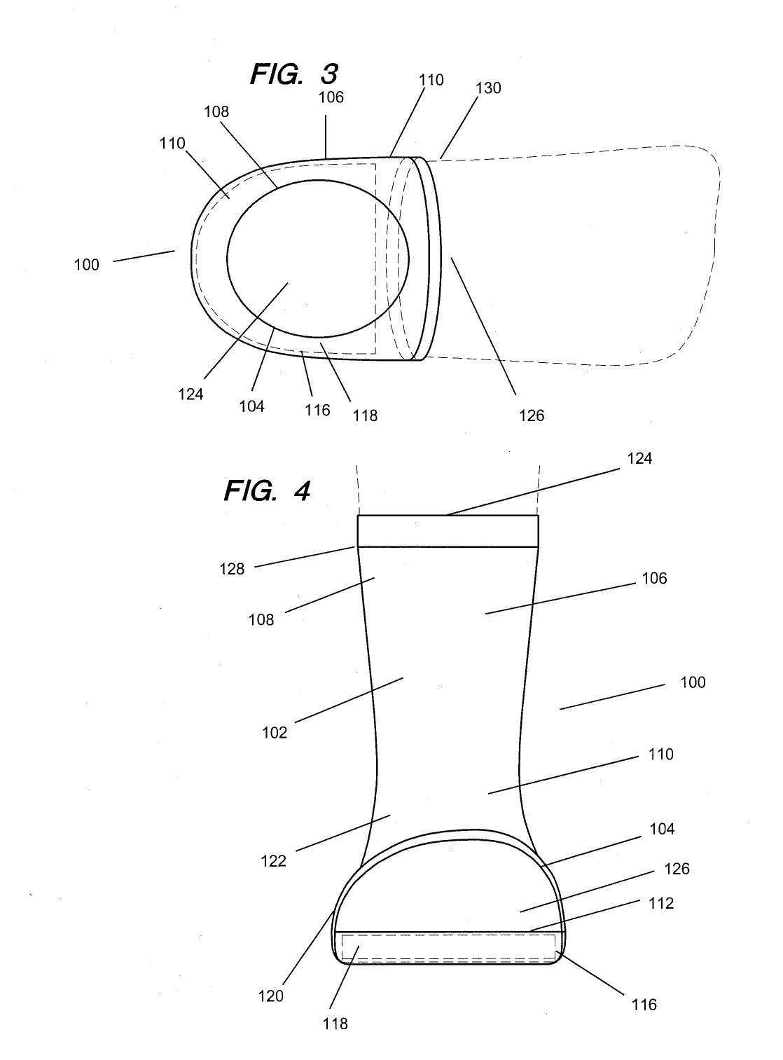

The tubular main body 102 may be constructed of an elastic material, in part or in whole, and sized so as to provide selective compression over the wearer's foot, ankle and/or calf In this manner, the invention offers the benefits of a compression sock or bandage as well as providing a simple, durable, hygienic and cost-effective means of securely holding an orthopedic support device in place. As shown in FIG. 3, the second end 110 of the embodiment contemplated in these Figures does not extend to cover the arch of the wearer's foot 130. In alternative embodiments, the second end 110 may so extend to cover the arch 130 and, in such an embodiment, the tubular main body 102 may be reinforced to provide support for the arch of the wearer's foot.

What is claimed, is:

1. An orthotic sock comprising:
a) a tubular main body forming an interior and an exterior surface and having an open first end and a second end;
b) an internal pocket located internal to said main body; and
c) an orthotic heel lift;
wherein said pocket substantially encloses said orthotic heel lift.

2. The orthotic sock of claim 1, wherein said first end is sized to snugly fit over a wearer's ankle and said second end is sized to snugly fit over at least a portion of said wearer's foot.

3. The orthotic sock of claim 1, wherein said orthotic heel lift is removably secured within said pocket.

4. The orthotic sock of claim 1, wherein said orthotic heel lift is adjustable.

5. The orthotic sock of claim 1, wherein said pocket is located so as to hold said orthotic heel lift against a wearer's heel.

6. The orthotic sock of claim 1, wherein said pocket is comprised of elastic material.

7. The orthotic sock of claim 1, wherein said main body is composed of elastic material.

8. The orthotic sock of claim 1, wherein said pocket is formed between said interior surface and a flap of material connected to said interior surface.

9. The orthotic sock of claim 8, wherein said main body has an opening at said second end.

10. The orthotic sock of claim 1, wherein said first end extends at least about 1 inch along said wearer's ankle region.

11. The orthotic sock of claim 10, wherein said first end extends at least about 2 inches along said wearer's ankle region.

12. The orthotic sock of claim 11, wherein said first end extends at least about 3 inches along said wearer's ankle region.

13. The orthotic sock of claim 12, wherein said first end extends over said wearer's entire ankle region.

14. The orthotic sock of claim 13, wherein said first end extends over at least a portion of said wearer's calf region.

15. The orthotic sock of claim 14, wherein said main body is dimensioned to provide compression over said wearer's calf region.

16. The orthotic sock of claim 1, wherein said main body is dimensioned to provide compression over said wearer's foot region.

17. The orthotic sock of claim 1, wherein said main body further provides support to said wearer's arch.

18. The orthotic sock of claim 1, wherein said main body is dimensioned to provide compression over said wearer's ankle region.

19. The orthotic sock of claim 1, wherein said orthotic heel lift is in part composed of foam rubber.

20. The orthotic sock of claim 1, wherein at least part of one surface of said orthotic heel lift is textured.
